(19) United States
(12) Patent Application Publication
(10) Pub. No.: US 2004/0241132 A1
(43) Pub. Date: Dec. 2, 2004

(54) Cosmetic

(75) Inventors: Dariush Majlessi, Toronto (CA); Manuela Marcheggiani, Toronto (CA)

Correspondence Address: REISING, ETHINGTON, BARNES, KISSELLE, P.C., P O BOX 4390, TROY, MI, 48099-4390, US

(73) Assignee: Isomers Laboratories, Toronto (CA)

(21) Appl. No.: 10/638,879

(22) Filed: Aug. 11, 2003

(30) Foreign Application Priority Data
May 30, 2003 (CA) 2,430,541

(51) Int. Cl. A61K 7/06

(52) U.S. Cl. 424/74

(57) ABSTRACT

A cosmetic comprised of a carrier-matrix comprised of a substantially uniformly viscous liquid extract of flax seed is provided. At least one deliverable ingredient is substantially evenly dispersed throughout the flax seed carrier-matrix. The liquid extract of flax seed can include linum usitatissimum (linseed) seed extract. In one embodiment, the deliverable ingredients can be hydrophilic. In another embodiment, the deliverable ingredients can be either hydrophobic or hydrophilic.

### FIELD OF THE INVENTION

The present invention relates generally to cosmetics and more particularly relates to cosmetics for preservation or restoration of the normal state of skin, or for the amelioration of environmental impact on the skin.

### BACKGROUND OF THE INVENTION

Environmental factors such as dryness, wind, extreme temperatures, and ultraviolet light can lead to irritated skin that is uncomfortable and less-healthy. Aging typically exacerbates these problems. Other types skin irritations and problems can also result from environmental factors and aging. Skin cosmetics are a very common way to help reduce and/or treat skin irritations and/or otherwise contribute to an overall desired effect on skin.

As is well understood by those of skill in the art, such cosmetics typically include a carrier-matrix for carrying one or more deliverable ingredients. The cosmetic may also have one or more additional ingredients such as preservatives or antioxidants. Or other ingredients that may increase the stability or shelf life of the product eg. Chealating agents.

Examples of well-known carrier-matrices include oils, water, waxes, thickeners, emulsions (i.e. silicone, oil-in-water or water-in-oil) and/or combinations thereof. In general, carrier-matrices are chosen so that they will hold whichever desired deliverable ingredients are present in the cosmetic in a manner that substantially evenly distributes the deliverables throughout the entire cosmetic, and maintains that distribution throughout the shelf-life of the cosmetic. Carrier-matrices are also selected so that they have a pleasing texture and feel on the skin.

Deliverable ingredients in cosmetics can include active ingredients and/or cosmetic ingredients and/or other ingredients that contribute the overall desired effect of the cosmetic in the formulation and on the skin. Examples of deliverable ingredients include emollients, which soften skin, and moisturizers, which function as a moisture barrier or to attract moisture from the environment or other anti-aging ingredients that may reduce the appearance of lines and wrinkles. Examples of moisturizers include dimethicone silicone, isopropyl lanolate, myristate, palmitate, lanolin, octyl dodecanol, oleic acid (olive oil), stearic acid and stearyl alcohol. Other types of deliverable ingredients will occur to those of skill in the art.

Additional ingredients include preservatives and antioxidants (including vitamins) which are used to reduce deterioration of the cosmetic. Examples of additional ingredients include trisodium and tetrasodium edetate (EDTA), tocopherol (vitamin E). Other types of additional ingredients will occur to those of skill in the art.

Further examples of carrier-matrices, deliverable ingredients and additional ingredients are well documented in the International Cosmetic Ingredient Dictionary and Handbook, 8^{th} edition 2000, published by The Cosmetic, Toiletry, and Fragrance Association, 1101 17th Street, NW, Suite 300, Washington D.C. 20036-4702, the contents of which are incorporated herein by reference. These ingredients are listed under an international nomenclature format known as “INCI”.

Typically, prior art carrier-matrices are primarily based on either oils or water, and other gelling/thickening agents, or combinations thereof. One problem with certain prior art oil carrier-matrices is that the oil can have undesired effects and interfere with the function of the deliverable ingredients—actually clogging skin pores and thereby interfering with the desired effects of the deliverable ingredients. In contrast, carrier-matrices based on water can fail to adequately suspend the deliverable ingredients. Another problem with water carrier-matrices is that they are eschewed by many consumers, who prefer that the cosmetic contain more deliverable ingredients. In addition, generally the commonly used prior art carrier-matrices do not directly posses skin benefits and only act as suspending agents, diluents, solvents, fillers.

One known deliverable ingredient is flax seed extract, also known as linum usitatissimum (linseed) seed extract. A process for obtaining flax seed extract is discussed in Canadian Patent 2167951, “Methods For Defibreing Of Flaxseed, Producing Flaxseed Kernels And Extracting Lignans And Water-Soluble Fibre From The Fibres” issued on Apr. 30, 2002, the contents of which are incorporated herein by reference. Flaxseed extract is available from Natunola Health, 21 Antares Drive, Unit 123-124, Nepean, Ontario, Canada K2E 7T8, and sold commercially as “Natunola® Flaxseed extract 160”. According Natunola, Natunola® Flaxseed extract 160 can be used as an emollient, film former, or skin conditioner. Flaxseed extract 160 can also act as an additional ingredient to a cosmetic as a viscosity building agent for aqueous systems, or as a viscosity controlling agent.

Like many deliverable ingredients, flax seed extract can be applied to skin in its pure form as a deliverable ingredient, however this is generally not desirable as over time the flax fibres therein can precipitate and thus it is generally preferred to use flax seed extract as a deliverable ingredient suspended within a carrier-matrix. Another problem with flax seed extract in its pure form is that it can have a non-uniform viscosity, making it unpleasing to the touch. It is thus typically preferred to use flax seed extract as a deliverable ingredient or as an additional ingredient.

Another known deliverable ingredient is flax seed oil, also known as linum usitatissiumum (Linseed) Seed Oil. It is known to use flax seed oil as a deliverable ingredient, but again, with results that are bounded by the limitations of prior art carrier-matrices.

### SUMMARY OF THE INVENTION

It is an object of the invention to provide a cosmetic that obviate or mitigates at least one of the above-identified disadvantages of the prior art.

An aspect of the invention provides a cosmetic comprised of a carrier-matrix comprised of a substantially uniformly viscous liquid extract of flax seed and at least one deliverable ingredient substantially evenly dispersed throughout the carrier-matrix.

The liquid extract of flax seed can include flax seed extract and the at least one deliverable ingredient can be hydrophilic. Where the carrier-matrix is flax seed extract, it can comprise greater than about 80% of the cosmetic, or the flax seed extract can comprise greater than about 90% of the cosmetic, or the flax seed extract can comprise greater than about 99% of the cosmetic. The flax seed extract can include of between about 0.1% to about 5% flax fibre, or the flax seed extract can include between about 1% to about 3% flax fibre, or the flax seed extract can include about 1.3% flax fibre.

The matrix-carrier can be enhanced with an oil soluble extract that is micro-encapsulated in the matrix-carrier for carrying one or more hydrophobic deliverable ingredients. Thus, the liquid extract of flax seed can include flax seed extract combined with a liposome of linum usitatissimum (linseed) oil extract, and the at least one deliverable ingredient can then be hydrophilic or hydrophobic.

The oil-soluble extract can be flax seed oil combined with lecithin. About five parts of the lecithin can be used for one part of the oil. The ratio of the oil soluble extract in the matrix-carrier is between about 1% to about 15%, or the ration can be between about 2% to about 10%, or the ratio can be about 5%.

The at least deliverable ingredient can be selected from the group consisting of matrixyl, oleyl alcohol, dioscorea villosa (wild yam) root extract, glycine soja (soybean) sterol, dimethicone silicone, isopropyl lanolate, myristate, palmitate, lanolin, octyl dodecanol, oleic acid (olive oil), stearic acid and stearyl alcohol. Other types of deliverable ingredients will occur to those of skill in the art.

The cosmetic can further comprise at least one additional ingredient. The additional ingredient can be selected from the group consisting of trisodium, tetrasodium edetate (EDTA), A-phenoxyethanol (CAS # 122-99-6), methylparaben (CAS # 99-76-3), butylparaben (CAS # 94-26-8), ethylparaben (CAS # 120-47-8), propylparaben (CAS # 94-13-3), B-Propylene Glycol, Diazolidinyl Urea, Methylparaben, Propylparaben, C-Methylparaben and Propylparaben, A-phenoxyethanol (CAS# 122-99-6), methylparaben (CAS#99-76-3), butylparaben (CAS#94-26-8), ethylparaben (CAS#120-47-8), propylparaben (CAS#94-13-3), B-Propylene Glycol, Diazolidinyl Urea, Methylparaben, Propylparaben, OR C-Methylparaben and Propylparaben.

Another aspect of the invention provides a method of formulating a cosmetic comprising the steps of: micronizing a non-uniformly viscous flax seed extract to produce a substantially uniformly viscous matrix-carrier; mixing at least one deliverable ingredient with the substantially uniformly viscous matrix-carrier to substantially uniformly disperse the at least one deliverable ingredient throughout the matrix-carrier.

The flax seed extract can include about 1.3% flax fibre.

The method can further comprising the step of, prior to the mixing step, mixing, using slow-agitation, a microencapsulated oil concentrate to the substantially uniformly viscous matrix-carrier. The microencapsulated oil concentrate can be comprised of flax seed oil mixed with lecithin.

A cosmetic comprised of a carrier-matrix comprised of a substantially uniformly viscous liquid extract of flax seed is provided. At least one deliverable ingredient is substantially evenly dispersed throughout the flax seed carrier-matrix. The liquid extract of flax seed can include linum usitatissimum (linseed) seed extract. In one embodiment, the deliverable ingredients can be hydrophilic. In another embodiment, the deliverable ingredients can be either hydrophobic or hydrophilic. The resulting cosmetic thus includes a carrier-matrix that is pleasing to the touch, and has desirable properties normally associated with deliverable ingredients, and also ready to carry such other deliverable ingredients as may be desired.

### BRIEF DESCRIPTION OF THE DRAWINGS

Embodiments of the present invention will now be described, by way of example only, with reference to the attached Figures in which:

FIG. 1 is a schematic representation of a carrier-matrix holding active ingredients in accordance with an embodiment of the invention;

FIG. 1a is a schematic representation of an enhanced carrier-matrix holding active ingredients in accordance with another embodiment of the invention;

FIG. 2 is a flow chart of a method for preparing an enhanced carrier-matrix in accordance with another embodiment of the invention; and,

FIG. 3 is a series of photographs showing results of applying a cosmetic of the present invention.

### DETAILED DESCRIPTION OF THE INVENTION

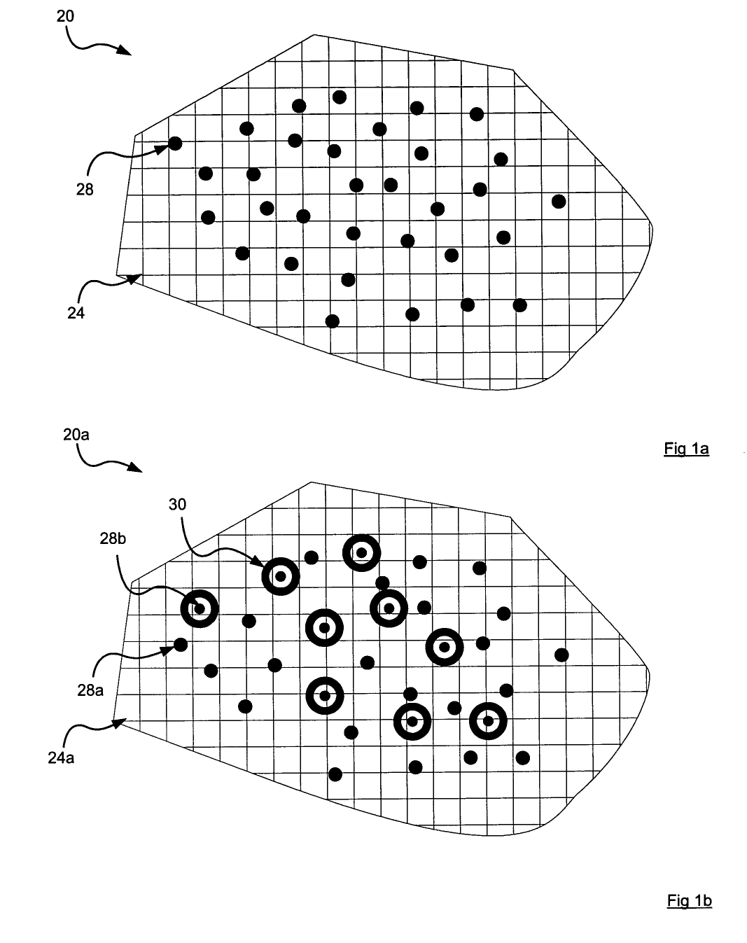

Referring now to FIG. 1, a cosmetic in accordance with an embodiment of the invention is indicated generally at 20. Cosmetic 20 includes a carrier-matrix 24 comprised of a substantially uniformly viscous liquid extract of flax seed and at least one deliverable ingredient 28 substantially evenly dispersed throughout the flax seed carrier-matrix 24. Carrier-matrix 24 can comprise greater than about 80% of cosmetic 20, or it can comprise greater than about 90% of the cosmetic, or it can comprise about 99% of cosmetic 20.

In one particular embodiment of the invention, the liquid extract of flax seed can be based on linum usitatissimum (linseed) seed extract, hereafter referred to as flax seed extract. Raw flax seed extract can be obtained from flax seed using any known commercially source. The active percentage of flax fibre in the flax seed extract is not particularly limited. However, the flax seed extract can be composed of about 0.1% to about 5% flax fibre, or it can be composed of about 1% to about 3% flax fibre, or, in a presently preferred format can be composed of about 1.3% flax fibre.

It can be desired that the flax seed extract is processed in a high speed and/or high sheer mixer, such as a homogenizer, at a rate of about 1000 to about 3000 RPM for an appropriate period of time in order to reduce precipitation of the flax fibre from the flax seed extract, and to attain a substantially uniformly viscous cosmetic carrier-matrix 24. This substantially uniform viscosity can also be obtained by other physical processing means known by those skilled in the art, such as milling or any other “micronizing” techniques. Where the percentage of flax fibre exceeds about 0.3% of the flax seed extract, then it is preferable to homogenize the flax seed extract to achieve the desired properties of carrier-matrix 24, or using other processing techniques that can achieve substantially the same result.

Deliverable ingredients 28 in the present embodiment are typically hydrophilic, as they are easily distributed throughout the flax seed extract carrier-matrix 24.

Referring now to FIG. 1a, a cosmetic in accordance with another embodiment of the invention is indicated generally at 20a. Cosmetic 20a includes a carrier-matrix 24a comprised of a substantially uniformly viscous liquid extract of flax seed and at least one deliverable ingredient 28a and 28b substantially evenly dispersed throughout the flax seed carrier-matrix 24a. In the present embodiment, the liquid extract of flax seed includes linum usitatissimum (linseed) oil extract (hereafter referred to as flax seed oil). Raw flax seed oil can be obtained from flax seed using a commercially available process. The flax seed oil is then combined with lecithin (or any other commercially available liposome basis) to produce a micro-encapsultion of flax seed oil which may be known as liposome, nanosome, or the like. The resulting micro-encapsulation in carrier-matrix 24 are indicated by reference 30a. Micro-encapsulation can also be created by other ingredients than lecithin.

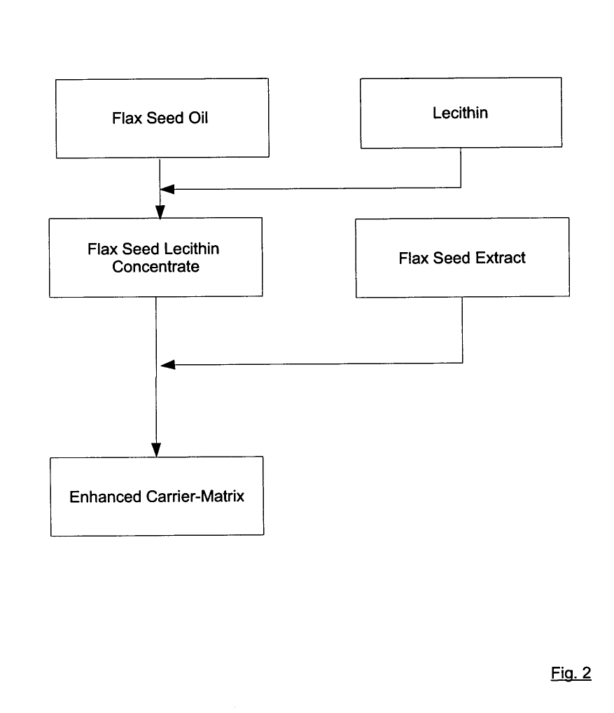

To better understand this embodiment, reference will now be made to the process shown in FIG. 2, wherein, lecithin is added (i.e. mixed or blended) to the flax seed oil to produce flax seed lecithin concentrate. About five parts of lecithin are used for one part oil.

Next, flax seed lecithin concentrate is added to the flax seed extract to enhanced carrier-matrix 24a. (The flax seed extract itself can be composed of various percentages of flax fibre, as described above.) Preferably, the concentration of flax seed lecithin concentrate in the enhanced carrier-matrix 24a can be anywhere between 0.1-15%, the remainder of the enhanced carrier-matrix 24a being flax seed extract. The enhancement results from microencapsulated flax seed oils, referred to herein for convenience as liposomes, which are identified at 30a in FIG. 1a. In order to ultimately form liposomes 30a in enhanced carrier-matrix 24a, the flax seed lecithin concentrate is typically added to the flax seed extract by means of slow agitation.

The deliverable ingredients 28a and 28b in the present embodiment can now be either hydrophilic or hydrophobic. Hydrophilic deliverable ingredients are indicated at 28a, while hydrophobic deliverable ingredients are indicated at 28b and contained within liposomes 30a. Hydrophobic deliverable ingredients 28b are thus emulsified within carrier-matrix 24a due to the presence of liposomes 30a.

Various types of hydrophilic deliverable ingredients 28 or 28a, and various types of hydrophobic can be used with the corresponding embodiments discussed above. Additionally, various types of additional ingredients can be added, as desired. Specific examples of how the embodiments discussed above can be implemented are provided below.

### EXAMPLE 1

Various cosmetic preparations were made according to the formulation of ingredients and accompanying ranges listed in Table I, which had a matrix-carrier for delivering hydrophilic deliverable ingredients.

TABLE I

| Ingredient Type | Ingredient | Percentage |
|---|---|---|
| Matrix-carrier | Flax Seed Extract | 92-99% |
| Deliverable Ingredient | Matrixyl | 1 to 8% |
| Additional Ingredient(s): | One or combination of | Total of 1% |
| (Preservative) | the following ingredients: | |
| | A-phenoxyethanol | |
| | (CAS# 122-99-6), | |
| | methylparaben(CAS#99- | |
| | 76-3), butylparaben | |
| | (CAS#94-26-8), | |
| | ethylparaben (CAS#120- | |
| | 47-8), propylparaben | |
| | (CAS#94-13-3), B- | |
| | Propylene Glycol, | |
| | Diazolidinyl Urea, | |
| | Methylparaben, | |
| | Propylparaben, OR C- | |
| | Methylparaben and | |
| | Propylparaben | |

### EXAMPLE 2

A specific cosmetic preparation was made according to the formulation of ingredients and accompanying ranges listed in Table II.

TABLE II

| Ingredient Type | Ingredient | Percentage |
|---|---|---|
| Matrix-carrier | Flax Seed Extract | 92% |
| Deliverable Ingredient | Matrixyl | 8% |
| Additional Ingredient(s): | methylparaben(CAS#99- | 1% |
| (Preservative) | 76-3) and propylparaben | |
| | (CAS#94-13-3) | |

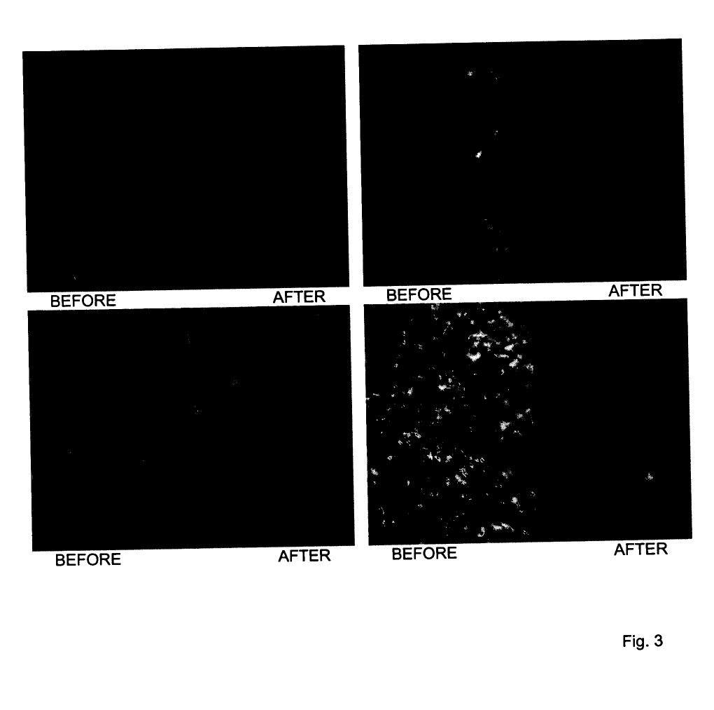

The cosmetic in FIG. 2 was applied to the skin of four subjects. A photograph of the subject's skin was taken prior to application of the cosmetic, and a photograph of the same portion of skin was taken subsequent to the application of the cosmetic. Desired results achieved included an appearance of reduced skin-dryness. Photographs of each subject are attached hereto in FIG. 3.

### EXAMPLE 3

Various additional cosmetic preparations were made according to the formulation of ingredients and accompanying ranges listed in Table III which had a matrix-carrier for delivering hydrophilic deliverable ingredients and hydrophobic deliverable ingredients.

TABLE III

| Ingredient Type | Ingredient | Percentage |
|---|---|---|
| Matrix-Carrier | Flax Seed Extract | 80-99% |
| Matrix-Carrier Enhancer | Soy Lecithin (Lecithin) | 1-10% |
| Matrix-Carrier Enhancer | Flax Seed Oil | 0.1-5% |
| Deliverable Ingredient | Soy Isoflavones | 0.1 to 20% |
| Deliverable Ingredient | Glycine Soja (Soybean) | 0.1-5% |
| | Germ Extract | |
| Deliverable Ingredient | Oleyl Alcohol | 0.1-5% |
| Deliverable Ingredient | Dioscorea Villosa (Wild | 0.1-5% |
| | Yam) Root Extract | |
| Deliverable Ingredient | Glycine Soja (Soybean) | 0.001-1% |
| | Sterol | |
| Additional Ingredient(s) | One or combination of the | Total of 1% |
| (Preservative) | following ingredients: | |
| | A-phenoxyethanol (CAS | |
| | # 122-99-6), | |
| | methylparaben (CAS # | |
| | 99-76-3), butylparaben | |
| | (CAS # 94-26-8), | |
| | ethylparaben (CAS # 120- | |
| | 47-8), propylparaben | |
| | (CAS # 94-13-3), B- | |
| | Propylene Glycol, | |
| | Diazolidinyl Urea, | |
| | Methylparaben, | |
| | Propylparaben, OR C- | |
| | Methylparaben and | |
| | Propylparaben | |

While only specific combinations of the various features and components of the present invention have been discussed herein, it will be apparent to those of skill in the art that desired subsets of the disclosed features and components and/or alternative combinations of these features and components can be utilized, as desired. For example, the order in which the above-identified ingredients are added is not limited to any particularly sequence.

The present invention provides a novel cosmetic having a matrix-carrier comprised of a substantially uniformly viscous liquid extract of flax seed for carrying deliverable ingredients to skin. In addition to conducting its function of carrying deliverable ingredients, the matrix-carrier itself can have desirable effects when applied to skin, thereby enhancing the overall cosmetic.

The above-described embodiments of the invention are intended to be examples of the present invention and alterations and modifications may be effected thereto, by those of skill in the art, without departing from the scope of the invention which is defined solely by the claims appended hereto.

1. A cosmetic comprised of a carrier-matrix comprised of a substantially uniformly viscous liquid extract of flax seed and at least one deliverable ingredient substantially evenly dispersed throughout the flax seed carrier-matrix.

2. The cosmetic according to claim 1 wherein the liquid extract of flax seed includes linum usitatissimum (linseed) seed extract.

3. The cosmetic according to claim 2 wherein the at least one deliverable ingredient is hydrophilic.

4. The cosmetic according to claim 2 wherein the liquid extract of flax seed includes linum usitatissimum (linseed) seed extract combined with a liposome of linum usitatissimum (linseed) oil extract.

5. The cosmetic according to claim 4 wherein the at least one deliverable ingredient is hydrophilic or hydrophobic.

6. The cosmetic according to claim 1 wherein the matrix-carrier is enhanced with an oil soluble extract that is micro-encapsulated in the matrix-carrier for carrying at one or more hydrophobic deliverable ingredients.

7. The cosmetic according to claim 1 wherein the carrier-matrix is flax seed extract and comprises greater than about 80% of the cosmetic.

8. The cosmetic according to claim 1 wherein the carrier-matrix is flax seed extract and comprises greater than about 90% of the cosmetic.

9. The cosmetic according to claim 1 wherein the carrier-matrix is flax seed extract and comprises greater than about 99% of the cosmetic.

10. The cosmetic according to claim 2 wherein the is flax seed extract includes of between about 0.1% to about 5% flax fibre.

11. The cosmetic according to claim 2 wherein the is flax seed extract includes between about 1% to about 3% flax fibre.

12. The cosmetic according to claim 2 wherein the is flax seed extract includes between about 1.3% flax fibre.

13. The cosmetic according to claim 6 wherein the oil-soluble extract is flax seed oil combined with lecithin.

14. The cosmetic according to claim 13 wherein about five parts of the lecithin are used for one part of the oil.

15. The cosmetic according to claim 6 wherein a ratio of the oil soluble extract in the matrix-carrier is between about 1% to about 15%.

16. The cosmetic according to claim 13 wherein the ratio is about 5%.

17. The cosmetic according to claim 15 wherein the oil soluble extract is comprised of lecithin and flax oil and the viscous liquid extract of flax seed is flax seed extract.

18. A method of formulating a cosmetic comprising the steps of:
micronizing a non-uniformly viscous flax seed extract to produce a substantially uniformly viscous matrix-carrier;
mixing at least one deliverable ingredient with the substantially uniformly viscous matrix-carrier to substantially uniformly disperse the at least one deliverable ingredient throughout the matrix-carrier.

19. The method according to claim 18 wherein the is flax seed extract includes between about 1.3% flax fibre.

20. The method according to claim 16 further comprising the step of, prior to the mixing step, mixing, using slow-agitation, a microencapsulated oil concentrate to the substantially uniformly viscous matrix-carrier.

21. The method according to claim 20 wherein the microencapsulated oil concentrate is comprised of flax seed oil mixed with lecithin.

22. The cosmetic according to claim 1 wherein the at least deliverable ingredient is selected from the group consisting of matrixyl, oleyl alcohol, dioscorea villosa (wild yam) root extract, glycine soja (soybean) sterol, dimethicone silicone, isopropyl lanolate, myristate, palmitate, lanolin, octyl dodecanol, oleic acid (olive oil), stearic acid and stearyl alcohol.

23. The cosmetic according to claim 1 further comprising at least one additional ingredient.

24. The cosmetic according to claim 1 wherein the additional ingredient is selected from the group consisting of trisodium, tetrasodium edetate (EDTA), A-phenoxyethanol (CAS # 122-99-6), methylparaben (CAS # 99-76-3), butylparaben (CAS # 94-26-8), ethylparaben (CAS # 120-47-8), propylparaben (CAS # 94-13-3), B-Propylene Glycol, Diazolidinyl Urea, Methylparaben, Propylparaben, C-Methylparaben and Propylparaben, A-phenoxyethanol (CAS# 122-99-6), methylparaben (CAS#99-76-3), butylparaben (CAS#94-26-8), ethylparaben (CAS#120-47-8), propylparaben (CAS#94-13-3), B-Propylene Glycol, Diazolidinyl Urea, Methylparaben, Propylparaben, OR C-Methylparaben and Propylparaben.
